(19) United States
(12) Patent Application Publication
(10) Pub. No.: US 2007/0100289 A1
(43) Pub. Date: May 3, 2007

(54) Methods of applying a substance

(76) Inventors: Irwin R. Berman, Jupiter, FL (US); Richard D. Gillespie III, Athens, TX (US); Gervasio Salgado, Marbella (ES)

Correspondence Address: J. Bruce Hoofnagle, P.O. Box 370, Lisbon, MD, 21765-0370, US

(21) Appl. No.: 11/595,018

(22) Filed: Nov. 10, 2006

Related U.S. Application Data
(62) Division of application No. 10/912,242, filed on Aug. 6, 2004.
Application No. 10/912,242 is a continuation-in-part of application No. 10/446,739, filed on May 29, 2003, now Pat. No. 7,125,394.
Application No. 10/446,739 is a continuation-in-part of application No. 10/160,166, filed on Jun. 4, 2002, now Pat. No. 7,141,036.

(51) Int. Cl. A61M 5/00

(52) U.S. Cl. 604/187

(57) ABSTRACT

An applicator 80b is attachable to a cream—containing cartridge 62 of a device 60 for administering the cream onto an area to be treated within a body cavity of a patient. An axial passage 82 is formed in a body 81 of the applicator 80b. A plurality of slots 204 are formed laterally and radially through the body 81, which are in communication with an intermediate portion 205 of the axial passage 82 and are contiguous with an outside surface of the body. A flange 226 extends radially from the outside surface of the body 81, and is formed with a contoured surface, which provides tactile notification when the applicator 80b has been inserted within the body cavity to a desired depth.

This application is a continuation-in-part of co-pending application Ser. No. 10/160,166, filed Jun. 4, 2002, which is incorporated herein by reference thereto.

### BACKGROUND OF THE INVENTION

This invention relates to an applicator and to methods of applying a substance, and particularly relates to an applicator for use with a focused dosimetry device for dispensing single and multiple doses of a medicinal cream, compound, or the like, for, and methods of, dispensing the cream from the applicator onto areas of the human anatomy to be treated.

Various medical conditions, which are located within the vaginal and/or anal cavities of the human anatomy, can be treated with medicinal creams and other substances of similar consistency. Frequently, such creams are prescribed by physicians. In some instances, it is beneficial and economical for the patient to self-administer, or have a caretaker administer, the measured dosage applications.

Applicators developed in the past facilitate the. dispensing of the cream generally within the vaginal and/or anal body cavities, but tend not provide structure which focuses the cream directly onto the critical areas for a most effective treatment.

Also, it is important to provide the self-administering patient, or caretaker, with notification when the applicator has been inserted an appropriate depth into the body cavity to a location which is adjacent the area to be treated.

Thus, there is a need for a multiple-dose delivery device, and an applicator thereof, which focuses the cream directly onto the critical areas to be treated, and which notifies the patient or the caretaker the applicator has been properly located.

### SUMMARY OF THE INVENTION

This invention contemplates an applicator for dispensing a substance therethrough, which includes a body formed about an axis and with an axial passage. The axial passage is formed with an entry opening at a first axial end thereof and a closed end at a second axial end of the axial passage opposite the first axial end. At least one slot has a prescribed slot length formed through the body and extends in an axial direction in communication with at least a portion of the axial passage. The at least one slot is spaced from the entry opening by a prescribed proximal distance. The body is formed with an outer surface which is contiguous with the at least one slot.

This invention further contemplates an applicator including a flange which extends from an outer surface of a body of the applicator laterally of the axial passage and has a surface facing toward a closed distal end of the body. The surface of the flange conforms to an area of the human anatomy surrounding an external opening of a body cavity intended for receipt of the body of the applicator.

This invention also contemplates an applicator which includes a flange for providing a tactile notification to the user of the applicator that a prescribed length of the applicator has been inserted into the body cavity.

Further, this invention contemplates a method of applying a substance to an area to be treated within a body cavity of a patient, including the steps of establishing a defined space adjacent the area to be treated within the body cavity at a location spaced from an external opening of the body cavity at least by a predetermined proximal distance, forming a mass of the substance externally of the body cavity, moving the mass of the substance into the defined space adjacent the area to be treated, and moving at least a portion of the mass of the substance from the defined space onto the area to be treated within the body cavity.

Also, this invention contemplates a method of applying a substance to an area to be treated within a body cavity of a patient by further including the step of tactilely notifying a user when the defined space has been established adjacent the area to be treated.

Other objects, features and advantages of the present invention will become more fully apparent from the following detailed description of the preferred embodiment, the appended claims and the accompanying drawings.

### BRIEF DESCRIPTION OF THE DRAWINGS

In the accompanying drawings:

FIG. 1 is a partially-sectioned side view showing a focused dosimetry device, having attached thereto a first embodiment of an applicator embodying certain principles of the invention;

FIG. 2 is an exploded side view showing components of the focused dosimetry device of FIG. 1, including the first embodiment of the applicator of FIG. 1 in accordance with certain principles of the invention;

FIG. 3 is a sectional side view showing a second embodiment of an applicator, in accordance with certain principles of the invention;

FIG. 4 is a sectional view taken along lines 4-4 of FIG. 3 showing internal details of the second embodiment of the applicator of FIG. 3, in accordance with certain principles of the invention;

FIG. 5 is a sectional view showing a third embodiment of an applicator, in accordance with certain principles of the invention;

FIG. 6 is a sectional view taken along line 6-6 of FIG. 5 showing the third embodiment of the applicator of FIG. 5, in accordance with certain principles of the invention; and

FIG. 7 is a perspective view showing a cap which can be attached to the focused dosimetry device of FIG. 1 in lieu of the applicators of FIGS. 1 through 6.

### DETAILED DESCRIPTION OF THE INVENTION

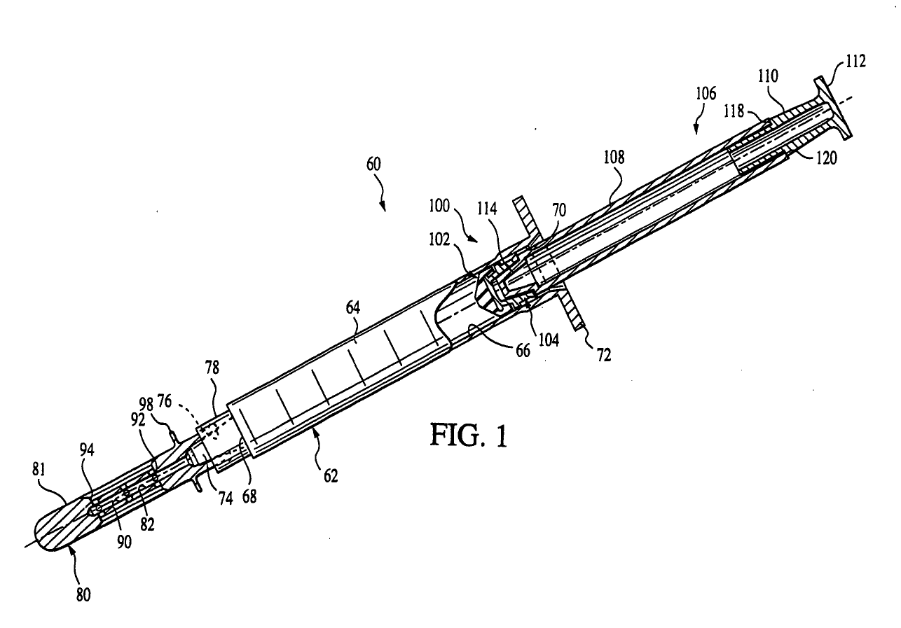

In the focused dosimetry device of FIG. 1, as described below, a substance, such as medicinal cream, compound, or the like, is deposited into a barrel of a cartridge of the device. The volume of the cream deposited in the cartridges of the respective embodiments represents a single dose or multiple doses.

The consistency of the cream is such that the cream does not flow easily within or out of the cartridge without a force being applied to the barrel-confined mass thereof. Typically then, a plunger head within the barrel is urged by pushing a stem, which is in contact with the head, to force the cream to exit the barrel.

The focused dosimetry device described below is particularly useful for applying and focusing each administered dose of cream to affected areas of vaginal and anal cavities of the human anatomy.

The below-described focused dosimetry device includes a dispensing end at which the cream is dispensed from the cartridge to the affected area of the patient. Such dispensing end of the focused dosimetry device will hereinafter be referred to as the distal end. The opposite end of each such device, which includes a stem, will be referred to as the proximal end of the device. The end of any component of each focused dosimetry device, which is closest to the distal end of the device, will be referred to as the proximal end of the component, and the other end of such component, which is opposite the proximal end, will be referred to as the distal end.

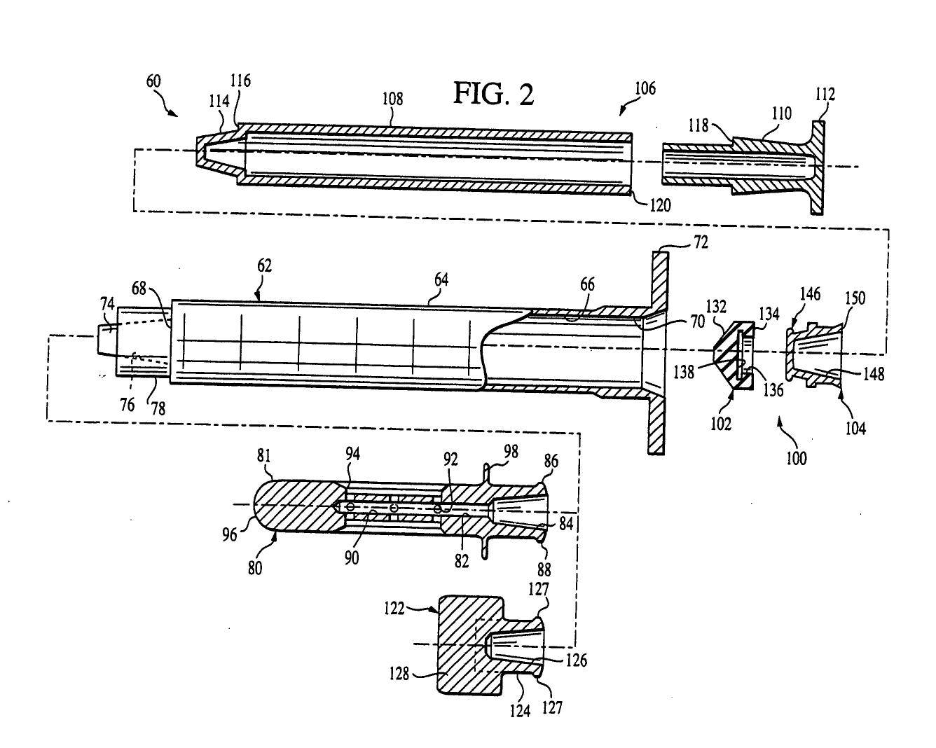

Referring to FIGS. 1 and 2, a focused dosimetry device is identified as device 60, and is typically used in multiple dose applications. The device 60 includes a cartridge 62, having a barrel 64, for containing a substance such as the medicinal cream, or any other substance having a cream-like consistency. The device 60 is a multiple dose device. However, the device 60 could be used as a single dose device.

In the device 60, the barrel 64 is formed with a hollow interior barrel passage 66, having a distal opening 68 at a distal end thereof and a proximal opening 70 at proximal end thereof. A finger-rest flange 72 is formed radially outward on the barrel 64 at the proximal end thereof.

A small-diameter sleeve 74 forms an integral part of the cartridge 62, and is in axial alignment with the barrel 64 at the distal end thereof, and forms a sleeve passage 76, which is in communication with the barrel passage 66. The exterior of the small-diameter sleeve 74 is tapered in the form of a frustum, with the smaller diameter of the frustum located at the distal end of the sleeve, and the axis of the frustum being coincidental with the axis of the barrel 64.

A large-diameter sleeve 78 also forms an integral part of the cartridge 62, at the distal end thereof, and is in axial alignment with the barrel 64, and coaxial alignment with the small- diameter sleeve 74. An internal cylindrical wall of the large- diameter sleeve 78 is threaded, and the proximal end of the sleeve is closed and not in communication with the barrel passage 66.

An applicator 80, or tip, which is formed with a smooth body 81 having an axial passage 82 therein, is assembled with the cartridge 62 at the distal end of the barrel 64, for example, by use of a known coupling facility such as the coupling facility identified with U.S. registered trademark LUER-LOC.

In particular, as shown in FIG. 2, the axial passage 82 of the applicator 80 is formed with a tapered proximal opening 84 which mates with the exterior taper of the sleeve 74 of the cartridge 62 to facilitate one aspect of the attachment of the applicator with the cartridge. The body 81 of the applicator 80 is formed with a first ear 86 and a second ear 88, which extend in radially opposite directions from the proximal end of the applicator. Upon assembly of the applicator 80 with the cartridge 62, the outboard ends of the ears 86 and 88 are threadedly applied to, and within, the large-diameter sleeve 78 by rotation of the applicator. The rotation of the applicator 80 also enhances the tapered assembly of the tapered small-diameter sleeve 74 with the tapered proximal opening 84 of the axial passage 82.

It is noted that facilities, other than as described above, can be used to attach the applicator 80 to the cartridge 62 without departing from the spirit and scope of the invention. Such attachment facilities could be threaded, unthreaded, tapered, press fit, or the like.

As shown in FIGS. 2 and 4, the applicator 80 is further formed with an inner passage 90, four axially-aligned, equally angularly spaced sets of three axially-spaced radially-oriented holes 92 in each set, and four axially-elongated slots 94. The tapered proximal opening 84, the inner passage 90, the holes 92 and the slots 94 of the applicator 80 are all in communication with each other to facilitate the smooth flow of the cream from the barrel 64 and through the applicator.

The applicator 80 is formed with a rounded distal end 96 and a tactile-indicator flange 98 near the proximal end thereof. The rounded distal end 96, and the smooth applicator body 81, provide a user-friendly applicator. The flange 98 provides a tactile indication to the self-administering patient, or the administering caretaker/user (hereinafter referred to as “the patient” or the “user,” respectively) that the applicator 80 has been inserted into the vaginal or anal cavity at the appropriate distance for placement of the slots 94 adjacent the areas to be treated with the cream.

Referring again to FIGS. 1 and 2, a plunger head 100 includes a compliant section, also referred to as a compliant plunger 102, which is locatable and movable within the passage 66 of the barrel 64. The plunger head 100 also includes a receptor section 104 which is coupled to the plunger 102. The plunger head 100 is assembled for sliding movement within the passage 66 of the barrel 64, between the proximal opening 70 and the distal opening 68 of the barrel.

A hollow rigid stem 106, which, for example, could be composed of a thermoplastic material such as polycarbonate, is formed by a secured assembly of a stem member 108 and a thumb piece 110, which is formed at the proximal end thereof with a flange-like thumb rest 112. The stem member 108 is formed with a stem structure 114 at a closed distal end thereof, with the stem structure being in the shape of a frustum. The distal end of the stem 106 is initially inserted into the proximal opening 70 of the barrel 64 such that the stem structure 114 engages a proximal end of the receptor section 104 of the plunger head 100. This provides facility for urging the plunger head 100 within the passage 66 of the barrel 64, toward the distal end thereof.

The diameter of the proximal end of the stem structure 114 of the stem 106 is smaller than the diameter of the stem member 108, thereby forming a shoulder 116 at the junction of the stem structure and the stem member. Also, a shoulder 118 is formed radially on an intermediate portion of the thumb piece 110, which abuts a proximal end 120 of the stem member 108 upon assembly of the stem member and the thumb piece, whereafter the thumb piece and the stem member are bonded together.

When the patient, or the user, is preparing to administer a dose contained in the cartridge 62, the cap 122 is removed and the applicator 80 is attached to the distal end of the cartridge. The distal end of the stem 106 is then inserted into the passage 66 of the barrel 64 at the proximal end thereof, and the stem structure 114 is moved into the receptor opening 148 to seat the stem structure in the opening.

The patient, or the user, then places the applicator 80 through an external opening of, and into, the body cavity to be treated. The patient, or the user, senses the initial engagement of the flange 98, with an area of the human anatomy adjacent the external opening, as tactile notification that the applicator 80 is in the appropriate location for administering the cream. The patient, or the user, pushes the stem 106 toward the distal end of the cartridge 62, whereby the first dose of cream is urged into the applicator 80, and is then dispensed from the applicator onto the area to be treated. By moving the device 60 away from the external opening of the body cavity, the applicator 80 is extracted from the body cavity.

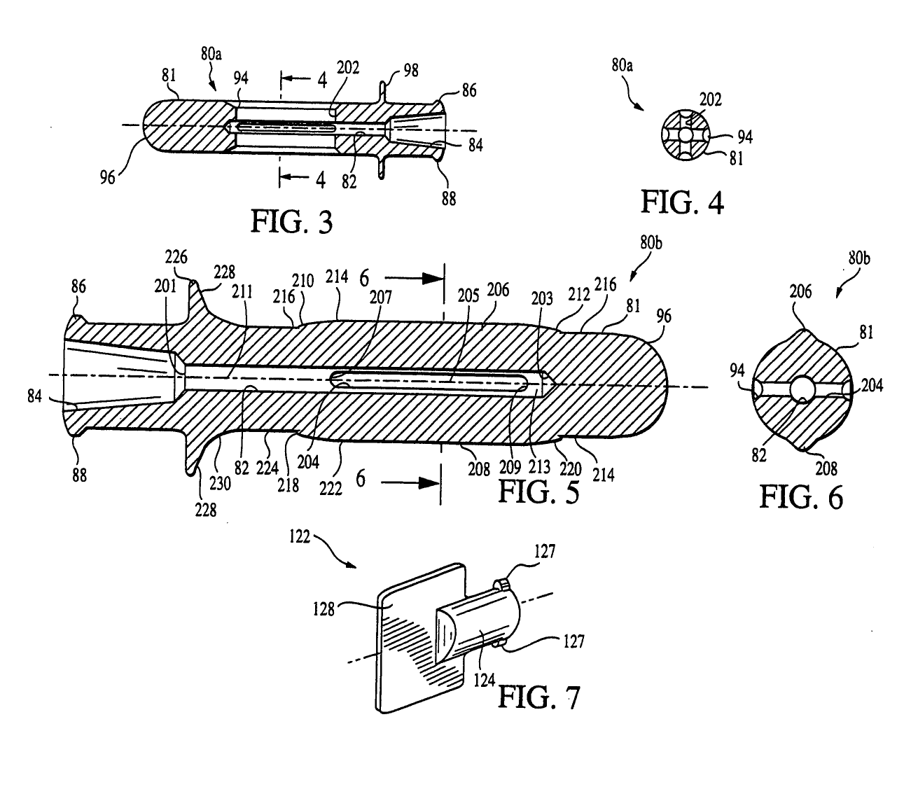

Referring to FIGS. 3 and 4, a second embodiment of an applicator, identified as applicator 80a, is formed with four equally, angularly and axially-elongated through slots 202 in place of the holes 62 and slots 94 of the applicator 80. Otherwise, the applicator 80a is identical to the applicator 80, and is assembled with the cartridge 62 in the same manner described above with respect to the applicator 80. The axially-elongated through slots 202 of the applicator 80a provide a faster and more widespread application of the cream to the affected area to be treated in comparison to the delivery through the holes 92 of the applicator 80.

Referring to FIGS. 5 and 6, a third embodiment of an applicator, identified as applicator 80b, and which is the preferred embodiment, is similar to the applicators 80 and 80a. For example, each of the applicators 80, 80a and 80b is formed with the smooth body 81 having the axial passage 82 therein, and is to be assembled with the cartridge 62 at the distal end of the barrel 64. Also, each of the applicators 80, 80a and 80b is formed with the axial passage 82, with a tapered proximal opening 84, which mates with the exterior taper of the sleeve 74 of the cartridge 62 to facilitate one aspect of the attachment of the applicators with the cartridge. The body 81 of each of the applicators 80, 80a and 80b is formed with the first ear 86 and the second ear 88, which extend in radially opposite directions from the proximal end of the applicators, to facilitate assembly of the applicators with the cartridge 62 in the manner described above with respect to the applicator 80.

In addition, the body 81 of the applicator 80b is formed with the two diametrically-opposed axially-elongated through slots 204, which, individually, are identical to the through slots 202 of the applicator 80a. The applicator 80b is also formed with two wipers 206 and 208 which extend radially outward from diametrically opposite sides of the body 81 of the applicator 80b, and which extend axially and parallel to the through slots 204. The through slots 204 and the two wipers 206 and 208 are equally angularly spaced about the axis of the body 81, as shown in FIG. 4. Each of the two wipers 206 and 208 extend radially outward from the body 81 in the shape of a convex mound.

A first pair of ramps 210 and 212 are formed with, and extend axially in opposite directions from, opposite ends of the wiper 206, and extend from a radially outwardmost surface 214 of the wiper to respective adjacent surface portions 216 of the body 81. A second pair of ramps 218 and 220 are formed with, and extend axially in opposite directions from, opposite ends of the wiper 208, and extend from a radially outwardmost surface 222 of the wiper to respective adjacent surface portions 224 of the body 81. It is noted that the applicator 80b can function in accordance with certain principles of the invention without the wipers 206 and 208, for example, in a manner similar to the applicator 80a.

The applicator 80b is also formed with a tactile-indicator flange 226 which functions in similar fashion as the flange 98 of the applicators 80 and 80a, as described above. A distal side of the flange 226 of the applicator 80b is formed with a straight portion 228, which extends toward the axis of the applicator and toward the distal end thereof. A shallow concave portion 230 of the flange 226 extends between the straight portion 228 and the outer surface of the body 81.

When a patient, or user, uses the applicator 80b, the body 81 is inserted into the body cavity of the patient until the patient, or user, tactilely senses engagement with the flange 226, in the manner noted above with respect to the use of the a applicators 80 and 80a. With the combined straight portion 228 and the concave portion 230 on the distal side of the flange 226, a gentile and gradual engagement of the flange with the patient is accomplished to avoid discomfort to the patient during the process.

In the same manner noted above with respect to the applicator 80a, the axially-elongated through slots 202 of the applicator 80b provide a faster and more widespread application of the cream to the affected area to be treated. After the cream has been applied within the body cavity, the focused dosimetry device 60, including the applicator 80b, may be rotated and/or oscillated about the applicator axis. With such action, the outer surface of the applicator 80b engages the deposited cream, and spreads the cream about the area to be treated.

While the applicator 80b is the preferred embodiment, any of the three applicators 80, 80a and 80b can be used with the cartridge 62, without departing from the spirit and scope of the invention.

As illustrated in FIG. 5, the body 81 of the applicator 80b is formed about a longitudinal axis, and with the axial passage 82. The axial passage 82 includes an entry port 201 at a proximal or first end of the axial passage. A dispensable substance, such as the above-noted cream, can be moved from the cartridge 62, through the entry port 201 and into the axial passage 82. The axial passage 82 extends axially longitudinally from the entry port 201 toward the closed distal end 96 of the body 81, and to a closed distal, or second, end 203 of the axial passage, by a predetermined passage distance. As noted above, each of the slots 204 is formed laterally or radially through the body 81, and is coincidental with an intermediate section 205 of the axial passage 82. Also, each of the slots 204 is in communication with the intermediate section 205 of the axial passage 82 and is contiguous with the outer surface of the body 81. This structural arrangement facilitates the dispensing of the cream from the intermediate section 205 of the axial passage 82 to an exterior area adjacent the outer surface of the body 81, and particularly onto the area to be treated within the body cavity of the patient.

Each of the slots 204 is elongated and extends axially longitudinally by a prescribed slot distance, or slot length, from a proximal end 207 of the slot to a distal or second end 209 of the slot. The pair of slots 204 represent a plurality of elongated circumferentially-spaced slots formed radially through the body 81 and extending in an axial direction in communication with the intermediate section 205 of the axial passage 82 and the exterior of the body.

A proximal section 211 of the axial passage 82 extends axially longitudinally by a prescribed proximal distance, or proximal length, from the entry port 201 to a location immediately adjacent the proximal ends 207 of the slots 204, but does not overlap the proximal ends 207. A distal section 211 of the axial passage 82 extends axially longitudinally by a prescribed distal distance, or distal length, from the closed distal, or second, end 203 of the axial passage 82 to a location immediately adjacent the distal ends 209 of the slots 204, but does not overlap the distal ends 209.

As further illustrated in FIG. 5, the proximal length of the axial passage 82 is less that the slot length of the slots 204, and is greater than the distal length of the axial passage.

The intermediate portion 205 of the axial passage 82, which is immediately adjacent and in communication with the slots 204, represents a defined space. In addition, the defined space can be provided by a container having an internal defined volume, with an entry opening for depositing the mass of the substance therein, and an exit opening for facilitating dispensing of at least portions of the mass of the substance therefrom. The defined space of the intermediate portion 205 of the axial passage 82, as well as the container, can also be considered an elongated defined space.

As also illustrated in FIG. 5, a proximal or first side of the tactile-indicator flange 226 is radially aligned with the entry opening 201 of the axial passage 82. Further, the angled straight portion 228 and the concave curved portion 230 of the distal or second side of the flange 226 blend to form a contoured surface. The contoured surface on the distal side of the flange 226 conforms anatomically to an area of the patient adjacent the external, or entry, opening of the body cavity, into which the applicator 80b is to be ultimately inserted.

In this manner, the contoured surface of the flange 226 forms a partially occlusive interface, or effectively a seal, with the anatomy of the patient to limit outflow leakage of the substance from the body cavity, at least during the period when the substance is being moved onto the area to be treated. Therefore, the contoured surface of the distal side of the flange 226 provides a means for limiting any outflow of the substance from the body cavity through the external opening thereof when at least portions of the substance are being moved through the opening of the container and onto the area to be treated.

The concave curved portion 230 of the flange 226 forms a curvature in the body 81 at a juncture of the outer surface of the body and the distal side of the flange, which is facing toward the closed distal end 96 of the body. As noted above, the body 81 is closed at the distal end 96 thereof, which prevents antegrade inflow leakage of the cream from the axial passage 82, or container, farther into the body cavity beyond the defined space, in a direction away from the external opening. It is noted that the body cavity could be an anal cavity, a vaginal cavity, or any cavity of the body in which the cream is to be administered.

The outer surface, or exterior, of the body 81 of the applicator 80b, as illustrated in FIGS. 5 and 6, is cylindrical in shape from the junction of the curved portion 230 of the flange 226 and the outer surface, and extends in the cylindrical shape to the distal end 96 of the body. In addition, the axial passage 82 is cylindrical in shape from the first end 201 to the second end 203 of the axial passage. It is noted that the outside surface and the axial passage of each of the applicators 80 and 80a, as illustrated in FIGS. 1 through 4, are also cylindrical in shape.

As noted above, the distal side of the flange 226, including the straight portion 228 and the curved portion 230, will tactilely engage the anatomical area of the patient, or the user, to notify the patient, or the user, that the slots 204 of the applicator 8b, or the exit opening of the container, have been located adjacent the area to be treated. Thus, the flange 226, and the distal side thereof, provide a means, responsive to locating the slots 204, or the exit opening of the container, adjacent the area to be treated by the patient, or the user, for notifying the patient, or the user, that each of the slots, or the exit opening of the container, is located adjacent the area to be treated.

When the patient is to self-administer, or a user is to apply, the cream from the applicator 8b onto the area of the body cavity to be treated, the proximal end of the applicator is assembled with the distal end of the cream-containing cartridge 62 by using any of the attachment techniques described above. Thereafter, the patient, or the user, inserts the closed, rounded, distal end 96 of the applicator 80b, or the container, through the external opening of the body cavity of the patient. The patient, or the user, continues to move trailing portions of the applicator, or the container, into the body cavity, whereby the applicator, or container, can move apart any interfacing portions of the body cavity which may be in the path of movement of the applicator, or container, into the body cavity.

Eventually, the defined space, as described above, is established at a location within the body cavity, which is spaced from the external opening of the body cavity at least by the prescribed proximal distance. The cream is formed in a mass externally of the body cavity such as, for example, in the cartridge 62. The mass of cream is then moved from the cartridge 62 and into the defined space of the axial passage 82 of the prepositioned applicator 80b, or the prepositioned container. Thereafter, at least portions of the mass of cream, adjacent the slots 204, or exit opening of the container, are moved, or urged, from the defined space onto the area to be treated within the body cavity.

The mass of the cream, which is moved into the defined space from the cartridge 62, is shaped in an elongated form of the elongated defined space, and is moved in a direction of elongation of the elongated form of the mass. Prior to moving the at least portions of the mass of the cream through the slots 204, or the exit opening of the container, the mass is maintained within the elongated defined space in the elongated form by the confinement of the applicator 80b, or the container.

The patient, or the user, continues to move the applicator 80b, or the container, into the body cavity until being tactilely notified by the flange 226, or similar structure of the container, that the slots 204, or exit opening of the container, have been placed adjacent the area to be treated.

As the patient, or the user, is tactiley notified, as described above, the distal or second side of the flange 226, or similar structure of the container, engages and compresses against the area surrounding the external opening of the body cavity. With this action, the partially occlusive interface is formed, effectively sealing the external opening of the body cavity during a period when the at least portions of the mass of the substance are being moved or urged onto the area to be treated. In this manner, the structure of the distal or second side of the flange 226, or similar structure of the container, limits the outflow of the substance from the body cavity during the moving or urging of the at least portions of the mass of the substance onto the area to be treated. After the cream, or substance, has been deposited onto the area to be treated, the patient, or the user, may rotate the applicator 80b, by rotating the cartridge 62 as described above, for providing a more uniform dispersal of the cream onto the area to be treated, prior to withdrawal of the applicator from the body cavity.

While the foregoing description has been directed toward the self-administration of the cream into the body cavity by the patient, as the user, it is to be understood that administering of the cream into the body cavity of the patient could be accomplished by the caregiver, or a user other than the patient. Therefore, the “user ” could be the patient or someone other than the patient. In the event that the user is someone other than the patient, the non- patient user would be tactilely notified when the contoured surface of the flange 226 engages the area around the external opening of the body cavity.

Further, as noted above, the applicators 80 and 80a include structure which functions in the manner of the applicator 80b. For example, as shown in FIGS. 1 through 4, applicators 80 and 80a are formed with the body 81 having the axial passage 82, and with slots which communicate with the axial passage. The slots of the applicators 80 and 80a are contiguous with the outer surface of the body 81, and each of the applicators is formed with a tactile flange which is radially aligned with the entry end of the axial passage 82.

The foregoing description has been directed to the use of the applicator 80b for the application of the medicinal cream onto areas to be treated within the body cavity of the patient. It is to be understood that the applicator 80b, as well as the applicators 80 and 80a, can be used for the application of a substance into a body cavity of the human anatomy for purposes other than medicinal. Further, the applicators 80, 80a and 80b can be used for applying a substance to surfaces other than those of the human anatomy. For example, the applicators could be used for applying a substance onto interior walls of a non-human enclosure having an entry opening. Further, apart from internal applications, the applicators 80, 80a and 80b may be used to apply the contents thereof to external surfaces of the human anatomy (e.g., skin) or to non-human external surfaces (e.g., industrial uses).

In general, the above-identified embodiments are not to be construed as limiting the breadth of the present invention. Modifications, and other alternative constructions, will be apparent which are within the spirit and scope of the invention as defined in the appended claims.

1-22. (canceled)

23. A method of applying a substance to an area to be treated within a body cavity of a patient, which comprises the steps of:
establishing a defined space adjacent the area to be treated within the body cavity at a location spaced from an external opening of the body cavity at least by a predetermined proximal distance;
forming a mass of the substance externally of the body cavity;
moving the mass of the substance into the defined space adjacent the area to be treated; and
moving at least a portion of the mass of the substance from the defined space onto the area to be treated within the body cavity.

24. The method as set forth in claim 23, which further comprises the step of:
forming the mass of the substance within the defined space in an elongated form.

25. The method as set forth in claim 24 which further comprises the step of:
moving the mass of the substance through the external opening of, and into, the body cavity in a direction of elongation of the elongated form of the mass.

26. The method as set forth in claim 24, which further comprises the step of:
maintaining the mass of the substance in the elongated form at least until the step of moving at least a portion of the mass of the substance onto the area to be treated within the body cavity.

27. The method as set forth in claim 23, wherein the body cavity is a vaginal cavity.

28. The method as set forth in claim 23, wherein the body cavity is an anal cavity.

29. The method as set forth in claim 23, wherein the body cavity is any cavity within the patient's body having an external opening and in which the substance is to be applied.

30. The method as set forth in claim 23, which further comprises the step of:
tactilely notifying the user when the defined space has been established adjacent the area to be treated.

31. The method as set forth in claim 23, which further comprises the step of:
sealing the external opening of the body cavity as the user is tactilely notified that the defined space has been established adjacent the area to be treated.

32. The method as set forth in claim 23, which further comprises the step of:
limiting the outflow of the substance from the body cavity through the external opening of, and from, the body cavity during the step of moving at least a portion of the mass of the substance from the defined space and onto the area to be treated.

33. The method as set forth in claim 23, which further comprises the step of:
preventing antegrade leakage from the defined space and farther into the body cavity in a direction away from the external opening.

34. A method of applying a substance to an area to be treated within a body cavity of a patient, where the area to be treated is spaced from an exterior opening of the body cavity by a prescribed proximal distance: which comprises the steps of:
providing a container having a defined space with a defined internal volume, an entry opening and an exit opening;
moving at least a portion of the container through the external opening of the body cavity to locate the exit opening of the container at least adjacent the area to be treated;
determining when the exit opening of the container is located adjacent the area to be treated;
locating a mass of the substance within the defined space of the container; and
urging at least portions of the mass of the substance from the container, through the exit opening thereof and onto the area to be treated.

35. The method as set forth in claim 34, wherein the step of determining is a tactile notification to a patient performing self-administering of the substance to the area to be treated.

36. The method as set forth in claim 34, which further comprises the step of;
effectively sealing the external opening of the body cavity at least during a period when the at least portions of the mass of the substance are being urged onto the area to be treated.

37. The method as set forth in claim 34, which further comprises the step of:
limiting the outflow of the substance from the body cavity through the external opening of the body cavity during the step of urging at least portions of the mass of the substance through the exit opening of the container and onto the area to be treated.

38. A method of applying a substance to an area to be treated within a body cavity of a patient, where the area to be treated is spaced from an exterior opening of the body cavity by a prescribed proximal distance: which comprises the steps of:
moving apart interfacing portions of the body cavity adjacent the area to be treated to form a defined space adjacent the area to be treated;
moving a mass of the substance into the defined space; and
moving at least portions of the mass of the substance from the defined space onto the area to be treated.
